(19) United States
(12) Patent Application Publication
(10) Pub. No.: US 2013/0211412 A1
(43) Pub. Date: Aug. 15, 2013

(54) IMPACTOR

(71) Applicants: Toby Hunt, West Sussex (GB); Andrew Taylor, Fishbourne (GB)

(72) Inventors: Toby Hunt, West Sussex (GB); Andrew Taylor, Fishbourne (GB)

(73) Assignee: FINSBURY (DEVELOPMENT) LTD, SURREY (GB)

(21) Appl. No.: 13/807,792

(22) PCT Filed: Jun. 3, 2011
(86) PCT No.: PCT/GB2011/051049

§ 371 Date: Mar. 7, 2013

(30) Foreign Application Priority Data
Jul. 1, 2010 (GB) 1011035.1

(51) Int. Cl. A61B 17/92

(52) U.S. Cl. CPC A61B 17/92
USPC 606/99

(57) ABSTRACT

An impactor (100) for transferring an impaction force to a prosthesis (4) coupled to a prepared distal end of a bone (6). The impactor (100) comprises a connector (154) arranged to couple to a prosthesis (4) and an impactor handle (134). The impactor handle (134) defines an impaction axis (110) extending into the prosthesis (4) such that an impaction force applied to the impactor handle (134) is transferred to the prosthesis (4) along the impaction axis (110). When the connector (154) is coupled to a prosthesis (4) the point at which the impaction axis (110) extends into the prosthesis (4) is spaced apart from the coupling between the connector (154) and the prosthesis (4).

The present invention relates to an impactor. In particular, embodiments of the present invention relate to a femoral impactor for coupling to a femoral prosthesis forming part of a replacement knee joint during implantation to transfer an impaction force to the prosthesis.

For a prosthesis, such as a replacement knee, it is commonly necessary to apply an impaction force to a prosthetic component in order to secure the prosthetic component in position. During knee surgery a femoral knee prosthesis is secured to a prepared end of a femur by applying an impaction force to the prosthetic component such that the prosthetic component bears down upon a prepared end of the femur forming an interference fit. Alternatively, bone cement may be provided between the prosthesis and the femur. It is important that the impaction force does not damage the articulating surface of the implant in order to ensure correct functioning of the implanted prosthesis. It is known to provide an femoral impactor having a first portion adapted to engage the implanted femoral implant and a coupled handle. An impaction force may be applied to the handle to drive the femoral prosthetic component into its final position.

Typically a femoral prosthesis comprises a shaped cup generally comprises lateral and medial condyles and anterior and posterior flanges arranged to fit over a shaped distal end of a femur. The condyles may be separate through the posterior portion and at least part of the distal portion of the cup and joined together with a recessed patella track through the anterior portion of the cup, extending to the distal portion of the cup. The space between the condyles, in particular through the distal portion of the prosthesis, is called the intercondylar notch. The patella track terminates at a curved lip within the intercondylar notch.

Conventional femoral impactors are arranged to couple to the distal portion of the cup, for instance by coupling to the condyles. In particular, conventional femoral impactors may comprise a shaped pad arranged to fit within the intercondylar notch and formed, for instance, from a plastics material such that the articulating surface of the prosthesis is not damaged. Hooks extend from the shaped pad laterally and medially to engage lateral and medial edges of the prosthesis. An impaction handle extends from the pad and defines an impaction axis which extends into the prosthesis between the hooks. The lateral and medial hooks comprise a connector for coupling the femoral impactor to the prosthesis, and the impaction axis extends through the connector.

After the distal femur has been appropriately shaped by resecting portions of the bone, the knee is hyper-flexed and the femoral prosthesis is fitted over the distal femur. The femoral impactor may securely couple to the femoral prosthesis such that the femoral impactor may be used to manipulate the prosthesis while it is positioned over the distal femur. Alternatively, the femoral prosthesis may only be coupled to the prosthesis after it is in place on the femur. Applying an impaction force to the end of the impactor handle transfers the impaction force through the impactor body to securely fit the prosthesis over the end of the femur, which may be through an interference fit, or secured with bone cement. The impaction axis is generally parallel to the longitudinal axis of the femur and extends into the femur through a distal resected surface.

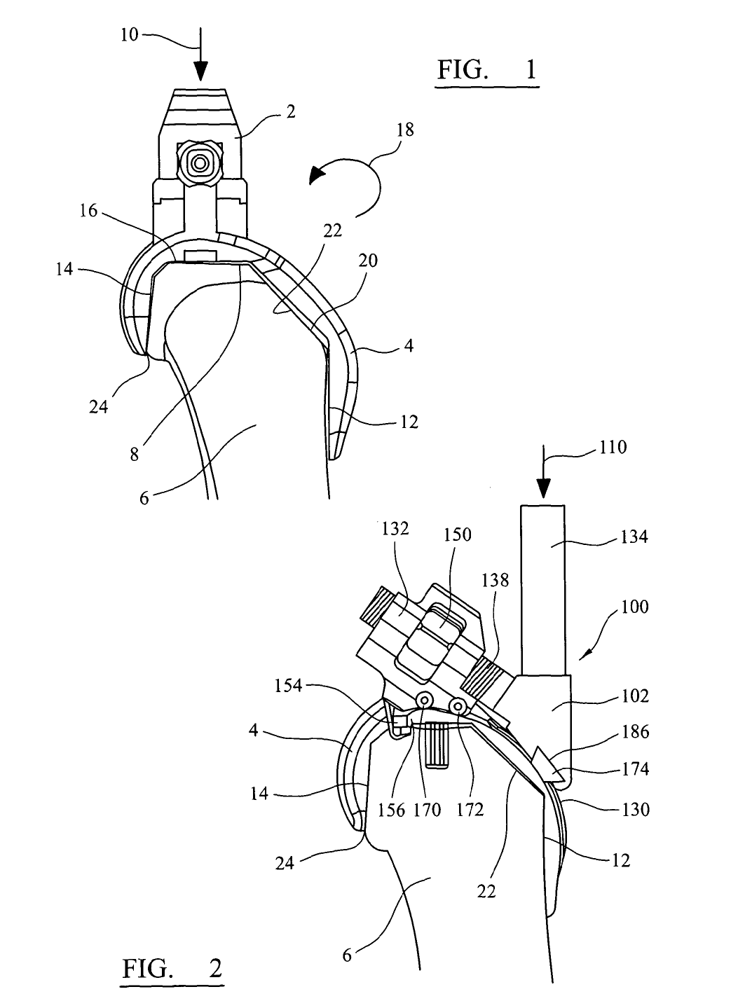

Referring to FIG. 1, this illustrates in a side view a conventional impactor body 2 coupled to a femoral prosthesis 4 which is seated over a prepared distal femur 6. It can be seen that the impactor body 2 is positioned generally over the resected distal femoral surface 8. The impactor handle is not shown coupled to the impactor body 2, however it will be understood that the impactor handle couples to the impactor body 2 generally normally to the resected distal femoral surface 8 such that an impaction force applied to the impactor handle is transmitted to the femoral prosthesis 4 along an impaction axis indicated by arrow 10 which extends through the coupling between the impactor body 2 and the femoral prosthesis 4 and into the resected distal femoral surface 8.

As can be seen in FIG. 1, the geometry of a conventional femoral prosthesis 3 is such that the anterior and posterior flanges 12, 14 are not parallel. As the impaction axis 10 is positioned closer to the posterior flange 14 (due to the asymmetry of the prepared femur and the prosthesis) the impaction force tends to cause the femoral prosthesis 4 to rotate posteriorly about the distal flange 16 as indicated by arrow 18. The interaction between the prosthesis and the differing sizes of respective resected surfaces of the femur causes differing amounts of resistance as the prosthesis is impacted, creating a force rotating the prosthesis anteriorly. The effect is to cause a gap 20 to open between the femoral prosthesis 4 and the bone anterior chamfer 22. Furthermore, bone may be compressed by the posterior flange 14 at point 24. It will be appreciated that the gap may be relatively small and will be difficult or impossible to observe as it is covered by the femoral prosthesis 4. However, the presence of a gap causes a reduction in the strength of the interface between the prosthesis 4 and the bone 6 and is therefore clearly undesirable. Gaps may only be formed for cementless implants, where an interference fit is necessary for a strong implant.

It is an object of embodiments of the present invention to obviate or mitigate one or more of the problems associated with the prior art, whether identified herein or elsewhere.

According to a first aspect of the present invention there is provided an impactor for transferring an impaction force to a prosthesis coupled to a prepared distal end of a bone, the impactor comprising: a connector arranged to couple to a prosthesis; and an impactor handle defining an impaction axis extending into the prosthesis such that an impaction force applied to the impactor handle is transferred to the prosthesis along the impaction axis; wherein when the connector is coupled to a prosthesis the point at which the impaction axis extends into the prosthesis is spaced apart from the coupling between the connector and the prosthesis.

An advantage of the first aspect of the present invention is that because the impaction axis is offset relative to the connector coupling the impactor to the prosthesis, the point at which the impaction axis extends into the bone can be chosen to counteract the effect of differing forces between the bone and the prosthesis across the surfaces of the prosthesis. This reduces the risk of the impaction causing rotation and misalignment of the prosthesis.

The impactor may further comprise an impactor body, the impactor handle being couplable to the impactor body and the impactor body being arranged to bear against a surface of a prosthesis such that an impaction force applied to the impactor handle is transferred to the prosthesis through the impactor handle.

The impactor may further comprise an adjustment mechanism arranged to couple the connector to the impactor body such that the distance between the connector and the impactor body can be adjusted.

The adjustment mechanism may comprises a size adjustment body including the connector, the size adjustment body comprising: a housing defining an adjustment bore arranged to receive a threaded rod extending from the impactor body; and a locking knob coupled to the housing and defining a threaded bore arranged to receive the threaded rod such that rotating the locking knob relative to the housing causes the threaded rod to slide into and out of the adjustment bore to adjust the distance between the connector and the impactor body.

The threaded rod may comprise at least one flattened side and the adjustment bore is non circular including at least one corresponding flattened side such that the threaded rod cannot rotate within the bore.

The connector may comprise a hook arranged to engage a lip formed within an intercondylar notch of a femoral prosthesis at the posterior end of the patella track.

The length of the hook extending from the housing of the size adjustment body may be such that when coupled to a femoral prosthesis the hook does not extend to the interior of the femoral prosthesis.

The hook may engage the prosthesis lip the femoral impactor is arranged to bear against the patella track such that the impaction axis extends into the prosthesis through the patella track proximal to the anterior flange of the prosthesis.

At least one contact pad may be coupled to the femoral impactor spaced apart from the hook such that the femoral impact contacts the prosthesis through the hook and the or each contact pad.

Said contact pad may be coupled to the impactor body such that the impaction axis passes through the contact pad to transfer the impaction force to the prosthesis through the contact pad.

The impactor may further comprise at least one further contact pad coupled to the size adjustment body such that the at least one further contact pad spaces the size adjustment body apart from the prosthesis other than contact between the size adjustment body and the prosthesis at the hook.

The connector may comprise a hook arranged to engage a lip formed within an intercondylar notch of a femoral prosthesis at the posterior end of the patella track, the impactor further comprising an impactor body integrally formed with the hook, the impactor handle extending from the impactor body, wherein the impactor body is arranged to bear against a surface of a prosthesis such that an impaction force applied to the impactor handle is transferred to the prosthesis through the impactor handle.

The impactor may further comprise an outrigger arranged to couple to the connector such that the outrigger extends from the connecter anteriorly when the impactor is coupled to a prosthesis, the outrigger defining at least one bore arranged to receive the impactor handle such that the impactor handle can extend through the bore along the impaction axis until a tip of the handle contacts the surface of the prosthesis.

According to a second aspect of the present invention there is provided a method of implanting a prosthesis, the method comprising: surgically preparing an end of a bone to receive a prosthesis; fitting the prosthesis over the prepared end of the bone; coupling an impactor the prosthesis, the impactor comprising a connector which couples to the prosthesis, and an impactor handle defining an impaction axis extending into the prosthesis, wherein the point at which the impaction axis extends into the prosthesis is spaced apart from the coupling between the connector and the prosthesis; and applying an impaction force to the impactor handle, the impaction force being transferred to the prosthesis along the impaction axis.

The present invention will now be described, by way of example only, with reference to the accompanying drawings, in which:

FIG. 1 is a side view of a conventional femoral impactor coupled to a femoral prosthesis in position over a prepared distal end of a femur;

FIG. 2 is a side and partially cross sectional view of a femoral impactor in accordance with a first embodiment of the present invention coupled to a femoral prosthesis in position over a prepared distal end of a femur;

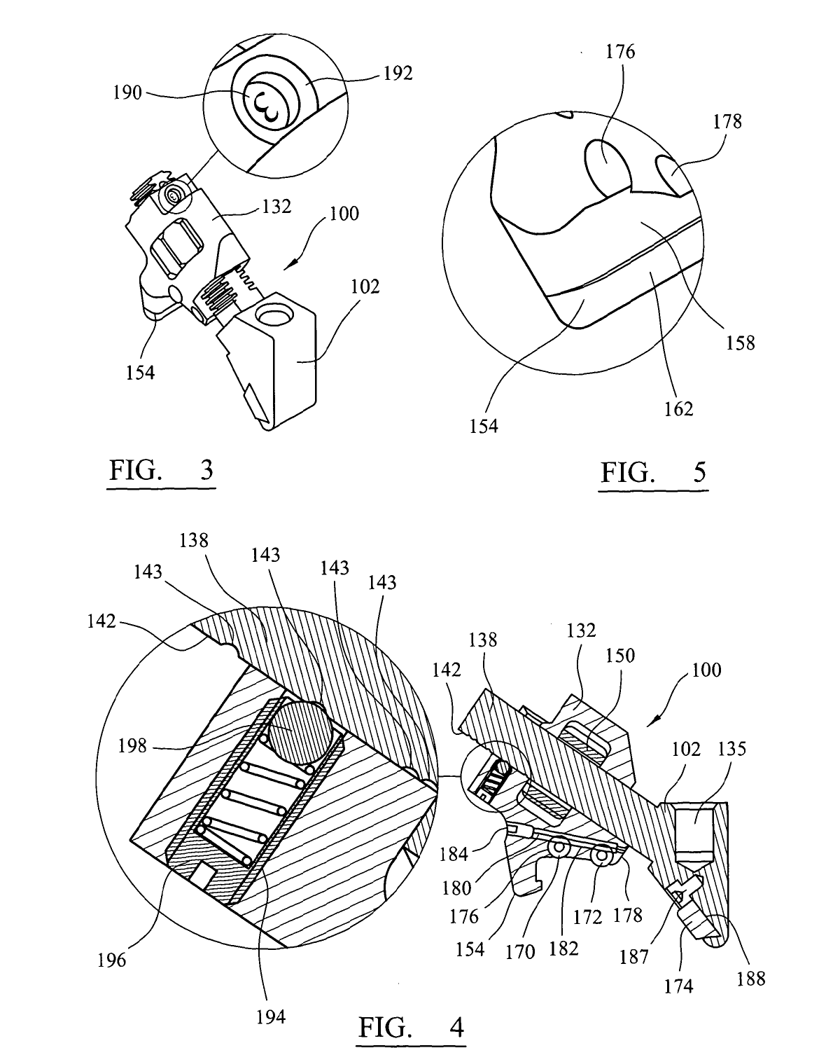

FIG. 3 is a perspective view of portions of the femoral impactor of FIG. 2, illustrating a size adjustment body and an impactor body coupled together, FIG. 3 including an enlargement showing detail of the size adjustment body;

FIG. 4 is a side view of portions of the femoral impactor of FIG. 2 illustrating a size adjustment body and an impactor body coupled together with a broken out section to show internal detail, FIG. 4 including an enlargement showing internal detail; and

FIG. 5 is an enlarged perspective view of part of the size adjustment body of the femoral impactor of FIG. 2;

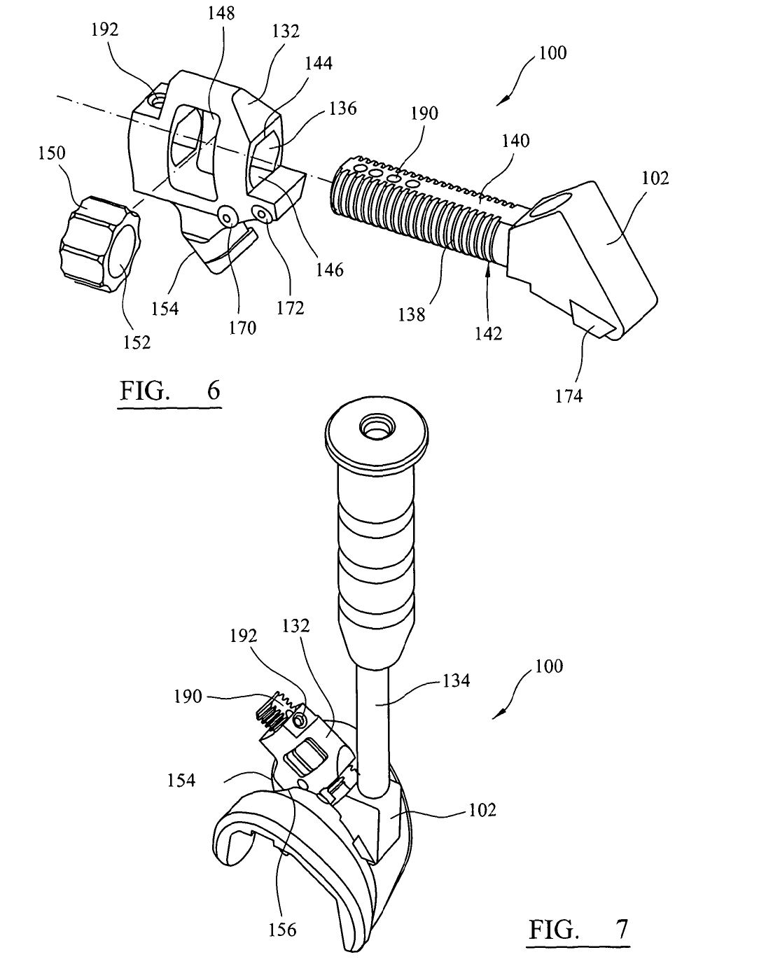

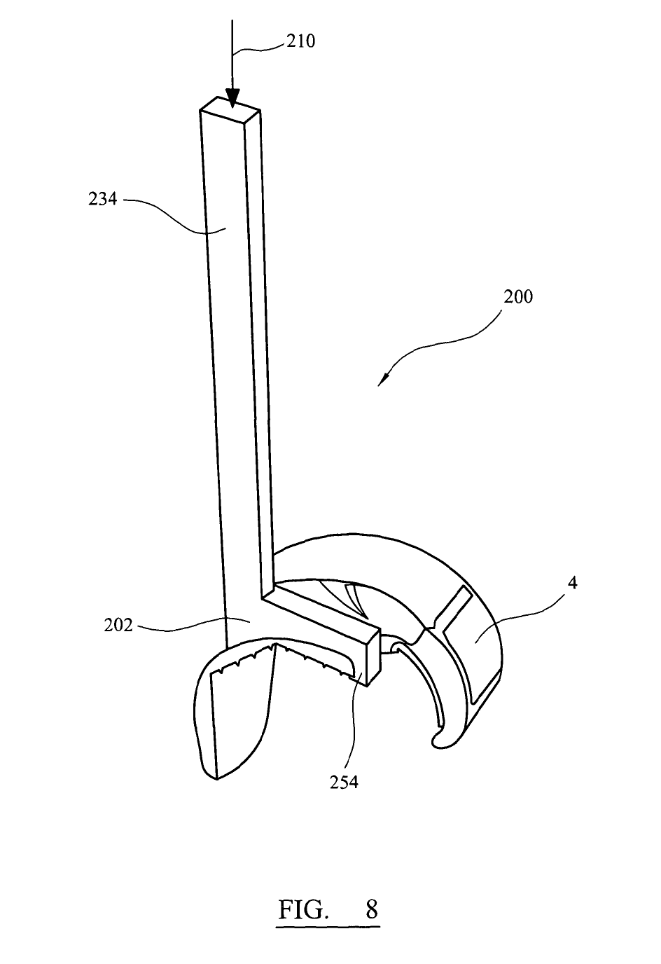

FIG. 6 is an exploded view of portions of the femoral impactor of FIG. 2, comprising the size adjustment body, impactor body and a sizing knob;

FIG. 7 is a perspective view of the femoral impactor of FIG. 2 coupled to a femoral prosthesis;

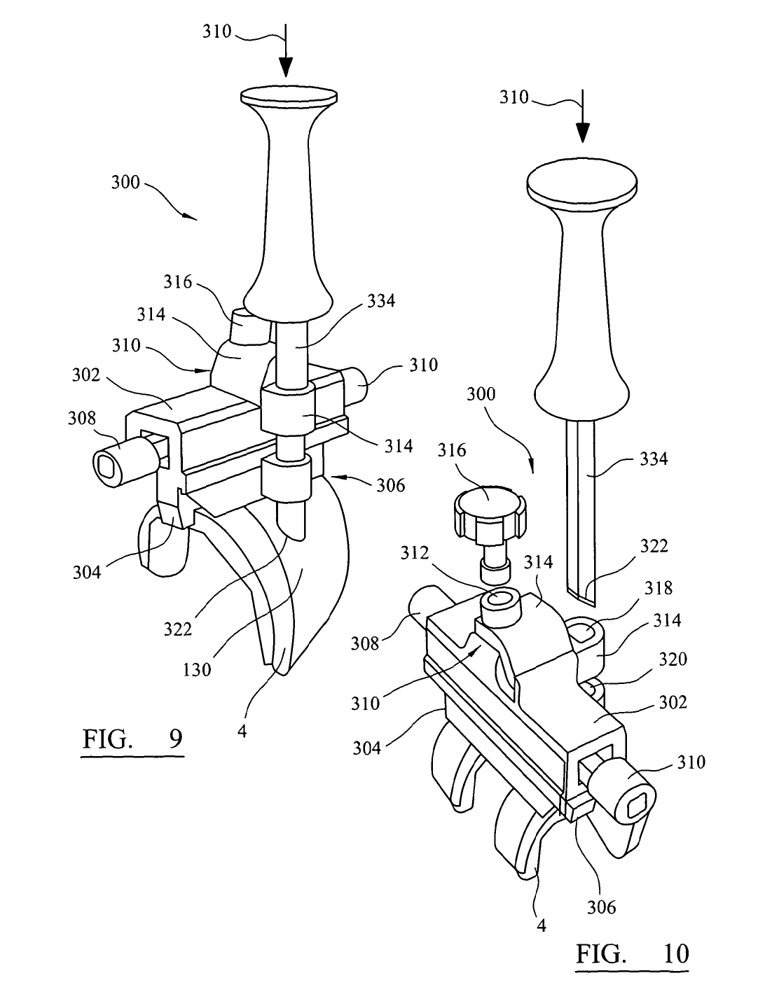

FIG. 8 is a perspective and partially cross sectional view of a femoral impactor in accordance with a second embodiment of the present invention coupled to a femoral prosthesis;

FIG. 9 is a perspective view of a femoral impactor in accordance with a third embodiment of the present invention coupled to a femoral prosthesis;

FIG. 10 is a partially exploded view of the femoral impactor of FIG. 9; and

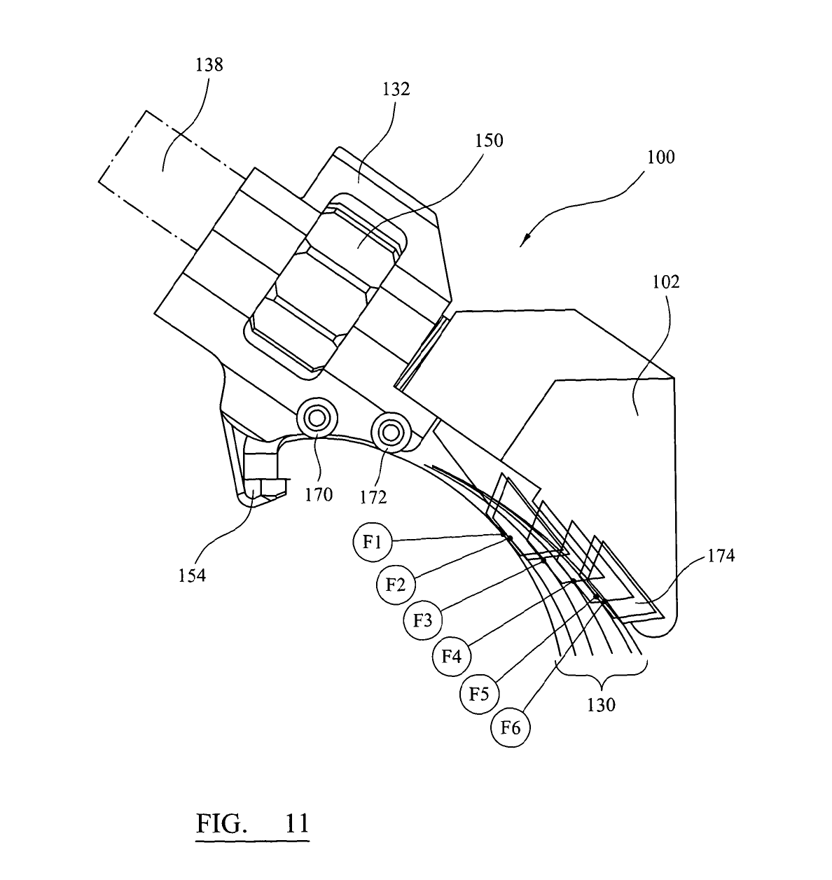

FIG. 11 is a side view of a plurality of femoral impactors according to FIG. 2 illustrating how the impactor adjusts in size to accommodate differing femoral prostheses.

Referring to FIG. 2, this illustrates in a side and partially cross sectional view a femoral impactor 100 in accordance with a first embodiment of the present invention coupled to a femoral prosthesis 4 in position over a prepared distal end of a femur 6. The femoral prosthesis 4 is partially cut away to illustrate the coupling between the femoral impactor 100 and the prosthesis 4. Specifically, the femoral prosthesis is cut away along the intercondylar notch and along the patella track to remove the left hand condyle.

The femoral impactor comprises an impactor body 102 which bears against the patella track 130, a size adjustment body 132 coupled to the impactor body 102 and an impactor handle 134. Only the lower part of the impactor handle 134 is illustrated. It can be seen that the impactor body 102 is positioned spaced apart anteriorly from the resected distal femoral surface 8. The impactor handle 134 is coupled to the impactor body 102 by being inserted into bore 135 (visible in FIG. 4), for instance by a threaded coupling or a tapered interference fit. The impactor handle 134 extends generally normal to the resected distal femoral surface 8 as is the case for the impactor of FIG. 1. However, in contrast to the impactor of FIG. 1, the impaction axis extending along the impactor handle 134 and indicated by arrow 110 is generally aligned with the anterior flange 12. An impaction force applied to the impactor handle 134 is transmitted to the femoral prosthesis 4 along an impaction axis indicated by arrow 110 which extends into the femoral prosthesis spaced apart from the point at which the size adjustment body 132 couples the femoral impactor 100 to the prosthesis 4.

The offset between the point at which the impaction axis 110 enters the prosthesis 4 and the coupling between the impactor 100 and the prosthesis 4 reduces the rotational effect caused by the fit between the femoral prosthesis 4 and the prepared femur 6. This reduces the tendency for a gap to open between the prosthesis 4 and the anterior chamfer 22. Additionally this reduces the tendency for bone to be compressed by the posterior flange 14 at point 24. It will be appreciated that in alternative embodiments the impaction axis 110 may not be exactly aligned with the anterior flange as the above described rotational force associated with the use of conventional impactors may be partially or fully overcome by a greater or smaller offset from the coupling between size adjustment body 132 and the prosthesis 4.

Referring also to FIG. 6, the impactor body 102 is coupled to the size adjustment body 132 such that the distance between the impaction axis 110 and the size adjustment body 132 is adjustable. The size adjustment body 132 comprises a housing 160 defining a through bore 136. The impactor body 102 comprises a threaded rod 138 arranged to be received within bore 136. Bore 136 is not threaded such that rod 138 may freely slide through the bore. Rod 138 has two flattened sides 140, 142 which interrupt the screw thread. Similarly, the bore 136 is not cylindrical and incorporates corresponding flattened sides 144, 146. Consequently, while rod 138 may freely slide through bore 136 the rod cannot rotate about its longitudinal axis within bore 136.

Size adjustment body 132 further comprises a cross bore 148 within housing 160 which cuts through bore 136 and is arranged to receive a sizing knob 150. Sizing knob 150 is arranged to fit within cross bore 148 and to receive the threaded rod 138 through a threaded bore 152. To couple the impactor body 102 to the size adjustment body 132 the sizing knob is inserted into cross bore 148 and the threaded rod 138 is inserted into bore 136 until it engages sizing knob threaded bore 152. Rotating the sizing knob 150 by hand causes the threaded rod 138 to slide within bore 136 to control the position of the impactor body 102 relative to the size adjustment body 132.

Size adjustment body 132 further comprises a connector to couple the femoral impactor to the prosthesis. The connector is in the form of a hook 154 arranged to engage lip 156 at the end of the patella track 130 within the distal part of femoral prosthesis 4. Hook 154 hooks underneath the distal part of the prosthesis 4, and the main part of the size adjustment body 132 sits above the prosthesis 4. The coupling between the size adjustment body 132 and the impactor body 102 is such that the threaded rod 138 extends away from the hook 154 and the impactor body 102 is spaced apart from hook 154. FIG. 5 illustrates an enlarged view of part of hook 154. It can be seen that the hook comprises a central curved portion 158 between the main housing 160 of the size adjustment body 132 and a flange 162 arranged to fit underneath the prosthesis. The gap between housing 160 and flange 162 is chosen to match the box wall thickness of the prosthesis at the lip 156 of the patella track 130. It will be appreciated that the size of the gap may not exactly match the thickness of the prosthesis at lip 156, which may vary according to the size of the prosthesis, so long as the gap is larger than the thickest prosthesis. The curvature of the central portion 158 corresponds to the curve of lip 156 when the prosthesis 4 is viewed from a distal position. It will be appreciated that in alternative embodiments there may be no flange 162 such that the hook 154 does not extend below the prosthesis. That is, in certain embodiments the hook 154 may not extend within the cup of the femoral prosthesis and so does not conflict with the bone.

Referring back to the side view of FIG. 2, it can be seen that when hook 154 engages lip 156, and in particular for the embodiment of FIG. 2, the flange 162 is received underneath lip 156, the sizing guide 132 bears against the prosthesis 4 at first and second cylindrical contact pads 170, 172. The impactor body 102 also bears against the prosthesis 4 at a trapezoidal contact pad 174. There are four points of contact between the femoral impactor 100 and the prosthesis 4: hook 154 which connects the impactor 100 to the prosthesis 4 and three pads 170, 172, 174 which allow the impactor to rest on the prosthesis 4. The contact pads bear against the patella track and are arranged to transfer a compressive load to the prosthesis, and in particular to transfer an impaction force to the prosthesis. The hook 154 is arranged to accurately position the impactor.

Cylindrical contact pads 170, 172 are received within channels 176, 178 formed within the underside of the size adjustment body 132 (best seen in the enlarged view of FIG. 5 with the pads removed). Cylindrical contact pads 170, 172 are formed from a material chosen to not damage the prosthesis bearing material, such as a plastics material, for instance polyoxymethylene. The cylindrical contact pads 170, 172 are reinforced with metal pins extending through the pads to increase the stiffness of the pads and to provide controlled deformation of the pads as the impactor is impacted against the prosthesis. Referring to FIG. 4, cylindrical contact pads 170, 172 are retain within channels 176, 178 by a metal wire 180 which extends through a bore 182 within the size adjustment body 132 and passes through the cylindrical contact pads 170, 172. The wire 180 is held in position within bore 182 by a grub screw which is received within an enlarged end 184 of bore 182.

Trapezoidal contact pad 174 is received within a corresponding recess 186 which extends to the edge of the impactor body 102 and thus allows the pad 174 to be inserted from the side. Pad 174 is retained within recess 186 by a cap head screw which is received in bore 187, though other ways of securing the contact pad 174 will be readily apparent to the appropriately skilled person. Contact pad 174 is positioned underneath the impactor body 102 such that it is between the impactor body 102 and the prosthesis 4. Specifically, the pad 174 is aligned with the impaction axis 110 such that when an impaction force is applied to the handle 134 the force is transmitted to the prosthesis 4 through the pad.

It will be appreciated that femoral prostheses are available in a range of sizes to accommodate differing sizes of femurs. In order to optimally position the impactor body 102, and therefore the impaction axis 110, sizing knob 150 is adjusted to slide rod 138 into or out of bore 136. As noted above, to reduce the risk of rotation of the prosthesis 4 as it is impacted into position, it may be desirable to generally align the impaction axis with the anterior flange 12 of the prosthesis 4. The chosen position for the impaction axis will vary according to the precise form of the prosthesis, and operating the sizing knob 150 will adjust the position of the impaction axis 110 for the selected size of that form of prosthesis. Additionally, adjusting the offset of the impaction axis ensures that the contact pads 170, 172, 174 are correctly positioned upon, and each in full contact with, the patella track 130.

To allow the surgeon to accurately adjust the offset of the impaction axis a gauge 190 is provided along the length of the upper flattened surface of rod 138, as is best visible in FIG. 6. Within the housing 160 of size adjustment body 132 there is provided an aperture 192 through which the gauge 190 can be viewed, as shown in FIG. 3, which also includes an enlargement of aperture 192. The numbers upon gauge 190 correspond to the range of sizes of femoral prosthesis. Rotating sizing knob 150 until the appropriate number is visible in aperture 192 allows the offset between hook 154 and impaction axis 110 to be set for the selected prosthesis.

Referring back to FIG. 4, the lower flattened surface 142 of threaded rod 138 may be provided with a series of transverse grooves 143 spaced apart along the longitudinal axis of the threaded rod 138. The spacing of the grooves corresponds to the variation in offset between the impaction axis 110 and the size adjustment body 132 for each size of femoral prosthesis. A further bore 194 is provided within the wall of the size adjustment body 132 proximal to hook 154 and arranged to receive a sprung ball grub screw 196. The sprung ball 198 is arranged to extend into bore 136 and to engage a transverse groove 143. The ball 198 is resiliently biased into bore 136. As sizing knob 150 is rotated the sprung ball progressively engages each transverse groove 143 as the threaded rod 138 slides through bore 136. When the ball 198 is engaged in a groove 143 this provides an increase in the resistance to further rotation of the locking knob 150 which can be detected by the surgeon. The sprung ball 198 encourages the position of the locking knob 150 to settle at rotational positions corresponding to each size of femoral prosthesis.

A method of using femoral impactor 100 will now be described. First, the required femoral prosthesis is determined and its size noted. The process of determining the required prosthesis falls outside of the scope of this specification. The chosen size of prosthesis is then used to adjust the impaction axis 110 offset by rotating sizing knob 150 until the corresponding number on gauge 190 is visible through aperture 192. Impaction handle 134 is then coupled to the impactor body 102, for instance by screwing the handle into a threaded bore within the impactor body. The impactor handle 134 may be a standard handle which is compatible with existing femoral impactors or other instruments. Furthermore, in some embodiments a slap hammer may be integrated with the handle. The prosthesis is then positioned over the shaped distal end of the bone by hand. The femoral impactor 100 is then coupled to the prosthesis by hooking hook 154 over lip 156 such that pads 170, 172, 174 rest against the patella track 130. A controlled impaction force is then applied to the handle 134 along the impaction axis 110, for instance using a free hammer or a slap hammer, to securely seat the prosthesis 4.

Referring now to FIG. 11, this illustrates side views of a plurality of impactors 100 coupled to prostheses of various sizes. The side views are superimposed such that the variation in the relative position of pads 170, 172, 174 upon the prosthesis patella track 130 can be seen. On the edge of patella track 130 is shown for each of a range of six differently size femoral prostheses labelled F1 to F6. It can be seen that each patella track 130 follows the same curve through its distal portion such that the size adjustment body 132 is seated upon the distal portion of each femoral prosthesis at the same position. Consequently, in the side view of FIG. 11 each size adjustment body is exactly aligned. It will be appreciated that as a consequence of this the size of contact pads 170, 172 can be chosen to conform to the distal part of each prosthesis and will be appropriately seated upon the prosthesis regardless of the size of prosthesis selected. Towards the anterior part of the prosthesis the patella track 130 curves differently according to the selected prosthesis. However, it is clear that if sizing knob 150 is turned to adjust the relative position of impactor body 102 the third contact pad 174 can be positioned to contact the patella track 130 such that all three contact pads rest upon the prosthesis surface when hook 154 engages the intercondylar notch. The contact pad 174 slides relative to the contact rollers 170, 172 along an axis parallel to the threaded rod 138. Careful selection of the spacing of grooves 143 and the position of the numbers on gauge 190 ensures that the contact pad is brought into contact with the patella track for each prosthesis. The particular spacing required is determined by the way in which each prosthesis within the range differs from the next biggest and smallest prostheses.

The femoral prosthesis may be disassembled in order to check its condition and to clean its parts by rotating sizing knob 150 to release the impactor body 102 and also to remove the sizing knob 150 from cross bore 148. If required, the contact pads may also be removed. While cleaning, the threads of rod 138 and locking knob 150 maybe inspected for damage and to remove burrs and other loose material. Cylindrical contact pads 170, 172 are intended to partially deform through use of the impactor to conform to the shape of the patella track 130. Excessive deformation causing the housing 160 to contact the prosthesis 4 is detectable by bruising of the material between the pads.

It will be appreciated that in alternative embodiments of the present invention the way in which the femoral impactor couples to the prosthesis, and the number and type of contact points (and contact pads) between the impactor and the prosthesis may vary. For instance, in place of a hook to engage the lip within the intercondylar notch the impactor may connect to lateral and medial edges of the condyles. In place of three contact pads spaced apart along the patella track in an anterior to posterior direction there may be more or fewer pads. For instance, for an impactor having a hook to couple to the lip the impactor body may be provided with two parallel contact pads arranged to contact anterior portions of the condyles. Advantageously this could provide a stable attachment to the prosthesis with three points of contact forming a tripod. For such an impactor an adjustment mechanism may not be necessary.

Referring now to FIG. 8 this illustrates an alternative femoral impactor 200 in accordance with a second embodiment of the present invention. The impactor 200 is shown coupled to a prosthesis 4, which has been partially cut away to illustrate the coupling. Impactor 200 comprises an integrally formed impactor comprising a handle 234, an impactor block 202 and a hook 254 formed as a single piece. The embodiment of FIG. 8 is much simpler than the embodiment of FIGS. 2 to 7 as there is no adjustment mechanism. However, the result is that the impaction axis 210 is at a fixed offset relative to hook 254, which reduces the effectiveness of reducing any potential rotation of the prosthesis 4 during implantation for varying sizes of prosthesis. Furthermore, as the impactor body 202 cannot be moved relative to the hook 254 the shape of the impactor facing the prosthesis cannot be varied to accommodate a range of sizes of prostheses. In order to prevent metal on metal contact with the prosthesis, it is likely to be necessary to provide larger or thicker contact pads (not illustrated in FIG. 8) or to exaggerate the curvature of the impactor body 202 facing the prosthesis thereby creating a larger gap between the impactor 200 and the prosthesis, other than at the contact pads.

Referring to FIGS. 9 and 10, these illustrate a femoral impactor 300 in accordance with a third embodiment of the invention. The femoral impactor 300 comprises an impactor body 302, which may be entirely conventional and is illustrated as being similar to that of FIG. 1. The conventional impactor body 302 comprises lateral and medial hooks 304, 306 to engage lateral and medial sides of the prosthesis 4 and adjustment screws 308, 310 to move hooks inwards and outwards to engage and release the prosthesis 4. The impactor body 302 may conventionally be used with an impactor handle coupled directly to the impactor body 302 by being received in a threaded bore 312 (not directly visible in FIG. 9, but visible in FIG. 10). However, in the embodiment of FIGS. 9 and 10 an outrigger 314 is coupled to the impactor body 302 and secured by knob 316 engaging bore 312. Outrigger 314 comprises at least one and preferably two aligned bores 318, 320 arranged to slidably receive an impaction handle 334 which extends downwards and terminates at a contact pad 322 which rests against the patella track 130 at a point which is spaced apart from the connector formed by hooks 304, 306. Applying an impaction force to the impaction handle 334 transfers an impaction force to the prosthesis 4 along impaction axis 310 which intersects the surface of the prosthesis 4 at a point which is spaced apart from the coupling between the impactor body 302 and the prosthesis 4.

Many of the components of the femoral impactors described above may be formed from metals, such as stainless steel, or other suitable biocompatible metals known for use in other surgical instruments. Parts intended to come into direct contact with articulating surfaces of the femoral prosthesis may be provided with contact pads formed from plastics or other materials which will not scratch the prosthesis. Alternatively portions of the femoral impactor may be coated or replaced with other materials which will not cause damage to the prosthesis.

While embodiments of the present invention have primarily been described above in connection with the surgical implantation of a femoral knee prosthetic component, it will be apparent to the skilled person that the invention is not limited to this application. More generally, the present invention is applicable to the implantation of any prosthesis where it is necessary to apply an impaction force to an articulating surface and where it is preferable for the axis along which the impaction force is applied to be offset relative to the point at which the impactor couples to the prosthesis. In particular, surgical impactors according to the present invention are applicable to any prosthetic implant which can be implanted by pushing against or applying an impaction force to part of an articulating or bearing surface with a stem which is offset relative to a resultant impaction axis. Alternatively, surgical impactors according to the present invention are applicable to any prosthetic implant which has an internal box geometry to receive a prepared portion of a bone which does not have at least two opposing contact points perpendicular to the implantation axis, or which has opposing contact regions which displace differing amounts of bone.

Further modifications to, and applications of, the present invention will be readily apparent to the appropriately skilled person from the teaching herein, without departing from the scope of the appended claims.

1. An impactor for transferring an impaction force to a prosthesis in contact with a prepared distal end of a bone, the bone having a longitudinal axis, the impactor comprising:
a connector couplable to the prosthesis at a first location; and
an impactor handle connected to the connector, the impactor handle having an impaction axis spaced apart from the first location along an axis perpendicular to the longitudinal axis.

2. The impactor of claim 1, further comprising an impactor body, the impactor handle being couplable to the impactor body, the impactor body being arranged to bear against a surface of the prosthesis such that an impaction force applied to the impactor handle is transferred to the prosthesis through the impactor handle.

3. The impactor of claim 2, further comprising an adjustment mechanism coupled to the connector and the impactor body, the distance between the connector and the impactor body being adjustable by the adjustment mechanism.

4. The impactor of claim 3, wherein the adjustment mechanism comprises a size adjustment body including the connector, the size adjustment body comprising:
a threaded rod;
a housing defining an adjustment bore arranged to receive the threaded rod extending from the impactor body; and
a locking knob coupled to the housing and defining a threaded bore arranged to receive the threaded rod such that rotating the locking knob relative to the housing causes the threaded rod to slide into and out of the adjustment bore to adjust the distance between the connector and the impactor body.

5. The impactor of claim 4, wherein the threaded rod comprises at least one flattened side and the adjustment bore is non circular including at least one corresponding flattened side such that the threaded rod cannot rotate within the bore.

6. The impactor of claim 4, claim 4, wherein the prosthesis is a femoral prosthesis having a lip formed within an intercondylar notch at the posterior end of the patella track, and the connector comprises a hook arranged to engage the lip.

7. The impactor of claim 6, wherein the length of the hook extending from the housing of the size adjustment body is such that when coupled to a femoral prosthesis the hook does not extend to the interior of the femoral prosthesis.

8. The impactor of claim 6, wherein, when the hook engages the lip, the femoral impactor is arranged to bear against the patella track such that the impaction axis extends into the prosthesis through the patella track proximal to the anterior flange of the prosthesis.

9. The impactor of claim 8, further comprising a contact pad coupled to the femoral impactor and spaced apart from the hook such that the femoral impact contacts the prosthesis through the hook and the contact pad.

10. The impactor of claim 7, wherein the contact pad is coupled to the impactor body such that the impaction axis passes through the contact pad to transfer the impaction force to the prosthesis through the contact pad.

11. The impactor of claim 10, further comprising a contact pad coupled to the size adjustment body such that the at least one further contact pad spaces the size adjustment body apart from the prosthesis other than contact between the size adjustment body and the prosthesis at the hook.

12. The impactor of claim 1, wherein the connector comprises a hook arranged to engage a lip formed within an intercondylar notch of a femoral prosthesis at the posterior end of the patella track, the impactor further comprising an impactor body integrally formed with the hook, the impactor handle extending from the impactor body, wherein the impactor body is arranged to bear against a surface of a prosthesis such that an impaction force applied to the impactor handle is transferred to the prosthesis through the impactor handle.

13. The impactor of claim 1, further comprising an outrigger arranged to couple to the connector such that the outrigger extends from the connecter anteriorly when the impactor is coupled to a prosthesis, the outrigger defining at least one bore arranged to receive the impactor handle such that the impactor handle can extend through the bore along the impaction axis until a tip of the handle contacts the surface of the prosthesis.

14. A method of implanting a prosthesis, the method comprising the steps of:
surgically preparing an end of a bone to receive a prosthesis;
fitting the prosthesis over the prepared end of the bone;
coupling an impactor the prosthesis at a first location, the impactor comprising a connector that couples to the prosthesis, and an impactor handle defining an impaction axis spaced apart from the first location along an axis perpendicular to the longitudinal axis; and
applying an impaction force to the impactor handle, the impaction force being transferred to the prosthesis along the impaction axis.

15. The impactor of claim 1, wherein the impaction axis is parallel to the longitudinal axis of the bone.
